SEQUENCE LISTING (3 sequences)

SEQ ID NO: 1 - DNA, 25 bp, Artificial Sequence
  Synthetic
tcgaacgttcgaacgttcgaacgtt

SEQ ID NO: 2 - DNA, 12 bp, Artificial Sequence
  Synthetic
cgacgttcgtcg

SEQ ID NO: 3 - DNA, 13 bp, Artificial Sequence
  Synthetic
cggcgccgtgccg

(19) United States
(12) Patent Application Publication
(10) Pub. No.: US 2022/0241321 A1
(43) Pub. Date: Aug. 4, 2022

(54) DIMERIC CPG OLIGONUCLEOTIDES FOR USE IN MODULATING IMMUNE RESPONSES

(71) Applicant: MICROBIO (SHANGHAI) CO., LTD., Shanghai (CN)

(72) Inventors: Yi-Chung CHANG, Shanghai (CN); Shih-Chi YEH, Shanghai (CN); Chih-Keng CHEN, Shanghai (CN); Chien-Hao CHANG, Shanghai (CN); Chu-Ying PENG, Shanghai (CN)

(21) Appl. No.: 17/610,000

(22) PCT Filed: May 8, 2020
(86) PCT No.: PCT/CN2020/089193

§ 371 Date: Nov. 9, 2021

(30) Foreign Application Priority Data
May 10, 2019 (CN) PCT/CN2019/086421

(51) Int. Cl. A61K 31/7125; A61K 9/08; A61P 35/00

(52) U.S. Cl. CPC A61K 31/7125; A61P 35/00; A61K 9/08

(57) ABSTRACT

Pharmaceutical compositions comprising a CpG oligonucleotide, a buffer agent, and one or more salts having a total salt concentration of about 80-130 mM. A majority population of the CpG oligonucleotides in the composition is in dimeric form. Also provided herein are uses of the pharmaceutical compositions for modulating immune responses in subjects in need of the treatment, for example, cancer patient.

### CROSS-REFERENCE TO RELATED APPLICATIONS

The present application is a U.S. National Stage Application under 35 U.S.C. § 371 of International Application No. PCT/CN2020/089193, filed on May 8, 2020, which in turn claims priority to, and the benefit of, International Application No. PCT/CN2019/086421, filed May 10, 2019, the entire contents of both of which are hereby incorporated by reference for all purposes.

### SEQUENCE LISTING

The application contains a Sequence Listing that has been filed electronically in the form of a text file, created Nov. 4, 2021, and named “112319-0024-7000US00_SUBSEQ.TXT” (943 bytes), the contents of which are incorporated by reference herein in their entirety.

### BACKGROUND OF THE INVENTION

CpG oligonucleotides are short synthetic nucleic acid molecules containing a “CpG” motif, in which C and G represent a cytidine residue and a guanosine residue, respectively, and “p” represents the phosphodiester linkage between the C and G residues. CpG oligonucleotides, when unmethylated, were found to activate stimulatory immune receptors (e.g., Toll-like Receptors or TLRs) on various immune cells, such as T cells or B cells, leading to the stimulation of innate immune responses.

It has been reported that local injection of CpG oligonucleotides to or nearby a tumor site would stimulate local anti-tumor immune responses, leading to reduction of tumor volume.

However, such anti-tumor activities are usually local. See, e.g., Rava et al., Science Translational Medicine, 10(426):eaan8723 (2018).

### SUMMARY OF THE INVENTION

The present disclosure is based, at least in part, on the unexpected discoveries that a CpG oligonucleotide in dimeric form successfully induced systemic anti-tumor immune responses upon local injection at a tumor site. As such, dimeric CpG oligonucleotides are expected to show systemic antitumor activity via local administration.

Accordingly, one aspect of the present disclosure provides a pharmaceutical composition comprising a CpG oligonucleotide of 5′-TCGAACGTTCGAACGTTCGAACGTT-3′ (SEQ ID NO: 1), a buffering agent, and one or more salts. The total salt concentration in the composition can be about 80-130 mM. At least 80% of the CpG oligonucleotides in the composition are in dimeric form.

In some embodiments, the CpG oligonucleotide can be modified. For example, the CpG oligonucleotide may comprise one or more phosphorothioate internucleotide linkage. In some examples, the CpG oligonucleotide is MBS513, which comprises the nucleotide sequence of SEQ ID NO: 1 and phosphorothioate internucleotide linkages, methylphosphonate linkages, or boranophosphate linkages.

The pharmaceutical composition comprises a buffer agent to stabilize the pH of the composition. Exemplary buffer agents include, but are not limited to, HEPES, DPBS or PBS. In some embodiments, the pharmaceutical composition may have a pH of 7-8.

Further, the pharmaceutical composition comprises one or more salts at a total concentration of about 80-130 mM. Exemplary salts for use in the present disclosure include, but are not limited to, KCl, NaCl, CaCl2), MgCl2, or a combination thereof.

Any of the pharmaceutical compositions disclosed herein may comprise a CpG oligodeoxynucleotide as disclosed herein (e.g., a dimeric form of SEQ ID NO:1) at a concentration of at least about 500 μM. In some instances, the CpG oligodeoxynucleotide can be at a concentration of about 500 μM to 5,000 μM (e.g., 500 μM to 2,000 μM).

Another aspect of the present disclosure relates to a dimeric oligonucleotide complex, comprising two CpG oligonucleotide molecules, at least one of which comprises the nucleotide sequence of SEQ ID NO:1. In some instances, the dimeric oligonucleotide complex can be a homodimer, in which both of the two CpG oligonucleotide molecules comprise the nucleotide sequence of SEQ ID NO:1.

In another aspect, the present disclosure provides a method for stimulating immune responses, comprising administering to a subject in need thereof an effective amount of any pharmaceutical compositions comprising the CpG oligonucleotide or the dimeric oligonucleotide complex as described herein. In some instances, the pharmaceutical composition or the dimeric oligonucleotide complex is administered to the subject by local injection, for example, intratumoral injection.

In some embodiments, the subject may be a human patient having, suspected of having, or at risk for a cancer. Exemplary target cancers include, but are not limited to, melanoma, colon cancer, lung cancer, breast cancer, liver cancer, and lymphoma. In some examples, the CpG oligonucleotide may be given to the patient at a dosage of 100 μg/kg to 4,000 μg/kg. Alternatively, the CpG oligonucleotide may be given to the patient at a dosage of about 40 nmol to about 150 nmol, e.g., about 50 nmol.

Also within the present disclosure are any of the dimeric CpG oligonucleotide complex or pharmaceutical compositions comprising such as described herein for use in treating any of the target diseases disclosed herein (e.g., cancer), as well as pharmaceutical compositions comprising the CpG oligonucleotide for use in manufacturing a medicament for cancer treatment.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIGS. 1A-1B are charts showing the property and function of CpG oligonucleotide MBS513. FIG. 1A is an image of DNA gel showing MBS513 are in dimeric form when dissolved in in SELEX buffer and in monomeric form when dissolved in distilled deionized water (ddH₂O). FIG. 1B is a chart showing the MBS513 activates TLR9 signaling in a dose-dependent manner.

FIGS. 2A-2E are charts showing the in vivo effect of MBS513 in tumor inhibition in a mouse model. FIG. 2A is a schematic illustration of an exemplary experimental design. FIG. 2B (local tumor) and FIG. 2C (distant tumor) are charts showing the effect of MBS513 on tumor volume. FIG. 2D and FIG. 2E are charts showing local (FIG. 2D) and distant (FIG. 2E) tumor inhibition by MBS513-1 and MBS513-2 (two different batches of MBS513, see Example 2 below) at 12.5 nmol, 25 nmol and 50 nmol.

FIG. 3 is a chart showing the dimer/monomer ratios of MBS513 under different concentrations as indicated.

### DETAILED DESCRIPTION OF THE INVENTION

CpG oligonucleotides (CpG DNA) are a class of agents that are capable of stimulating a potent, orchestrated immune responses, for example, anti-tumor immune responses. Local administration of CpG oligonucleotides showed promising results in reducing local tumor growth; however, such local administration usually would not lead to systemic immune response against tumors. See, e.g., Sagiv-Barfi et al., Science Translational Medicine, 10(426):eaan4488 (2018).

The present disclosure is based, at least in part, on the unexpected discoveries that proper salt concentration plays an important role in formation of dimeric CpG oligonucleotides and such dimeric oligonucleotides induced systemic immune responses against tumor cells when injected locally at a tumor site.

Accordingly, described herein are pharmaceutical compositions comprising a CpG oligonucleotide and one or more salts at a suitable total concentration such that majority of the CpG oligonucleotides (e.g., at least 80%) are in dimeric form. Also provided herein are uses of such pharmaceutical compositions for inducing immune responses, for example, systemic anti-tumor immune responses even when the pharmaceutical composition is administered at a local site (e.g., at a tumor site).

### I. Pharmaceutical Compositions Maintaining Dimeric Form of CpG Oligonucleotides

One aspect of the present disclosure provides a pharmaceutical composition that comprises a CpG oligonucleotide, a buffer agent and one or more salts. In some instances, the pharmaceutical composition may be an isotonic solution.

The total salt concentration in the composition can be about 80-130 mM. Such pharmaceutical composition may be capable of maintaining dimeric form of the CpG oligonucleotides contained therein, for example, at least 80% (e.g., at least 85%, 90%, 95%, 96%, 97%, 98%, 99% or above) of the CpG oligonucleotides are in dimeric form. As used herein, a dimer or a dimeric form refers to a nucleotide complex comprising two CpG oligonucleotide molecules. In some instances, a dimer or dimeric form as disclosed herein is a homodimer or homodimeric form, i.e., containing two identical oligonucleotide molecules such as those disclosed herein.

(i) CpG Oligonucleotides

“CpG” refers to a 5′ cytosine (“C”) and a 3′ guanine (“G”) linked by a phosphate bond (“p”). As used herein, the term “CpG oligonucleotides” refers to any CpG-containing oligonucleotide that is capable of activating an immune cell (immunostimulant). At least the C of the 5′ CpG 3′ must be unmethylated. CpG oligonucleotides may be prepared by chemical synthesis following routine technology or obtained from a commercial vendor.

In some embodiments, a CpG oligonucleotide used in the instant disclosure can be 20-100 nucleotides (nts) in length, (e.g., 25-100 nts). In some embodiments, a CpG oligonucleotide can be 25-100, 25-90, 25-80, 25-70, 25-60, 25-50, 25-40, or 25-30 nucleotides in length.

In some embodiments, the CpG oligonucleotides described herein may be DNA (CpG oligodeoxynucleotide or CpG ODN) molecules, DNA/DNA duplex, RNA molecules, or DNA/RNA hybrid molecules. In some examples, CpG oligonucleotides may be linear or circular. In some examples, CpG oligonucleotides may be partially circular or form a hairpin loop. In some examples, CpG oligonucleotides may be single stranded. In other examples, the CpG oligonucleotides may be double stranded.

CpG oligonucleotides described herein include CpG oligonucleotides having one or more modifications. Modifications include, but are not limited to, base modifications, sugar modifications, and backbone modifications. Such modifications may render the CpG oligonucleotide more stable and/or less susceptible to degradation under certain conditions. For example, in some embodiments, CpG oligonucleotides are nuclease-resistant.

CpG oligonucleotides of the present disclosure, in some embodiments, have a homogenous backbone (e.g., entirely phosphodiester or entirely phosphorothioate) or a heterogeneous (or chimeric) backbone. Phosphorothioate backbone modifications may render an oligonucleotide less susceptible to nucleases and thus more stable (as compared to a native phosphodiester backbone nucleic acid) under certain conditions. Other linkages that may provide more stability to a nucleic acid of the present disclosure include, without limitation, phosphorodithioate linkages, methylphosphonate linkages, methylphosphorothioate linkages, boranophosphonate linkages, peptide linkages, alkyl linkages and dephospho-type linkages. Thus, in some embodiments, CpG oligonucleotides have non-naturally occurring backbones. In some embodiments, CpG oligonucleotides have backbones that are entirely phosphorothioate. Any class of CpG oligonucleotide may be used as described herein. In some embodiments, a CpG oligonucleotide may be selected from the group consisting of a class A CpG oligonucleotide, a class B CpG oligonucleotide, and a class C CpG oligonucleotide.

Class A CpG oligonucleotides, in some embodiments, are characterized by the ability to induce high levels of interferon-α while having minimal effects on B cell activation. In some embodiments, class A CpG oligonucleotides may contain a hexamer palindrome GACGTC, AGCGCT, or AACGTT. Yamamoto et al. J Immunol 148:4072-6 (1992). In some embodiments, class A CpG oligonucleotides have poly-G rich 5′ and 3′ ends and a palindromic center region. In some embodiments, class A CpG oligonucleotides at the 5′ and 3′ ends have stabilized internucleotide linkages and the center palindromic region has phosphodiester linkages. In some embodiments, class A CpG oligonucleotides may lack one or more of the poly G ends and the palindromic center. In some embodiments, class A CpG oligonucleotides, may have all phosphorothioate or all phosphodiester internucleotide linkages. Class A CpG oligonucleotides have been described, for example, in PCT application WO 2001/022990, the relevant disclosures thereof are incorporated by reference for the purpose or subject matter disclosed therein.

Class B CpG oligonucleotides, in some embodiments, strongly activate human B cells but have minimal effects inducing interferon-α without further modification. In some embodiments, class B CpG oligonucleotides include the sequence 5′ X₁CGX₂ 3′, wherein X₁ is T, G or A; X₂ is T, C, or A. In some embodiments, class B CpG oligonucleotides that are fully stabilized and include an unmethylated CpG dinucleotide within certain preferred base contexts are potent at activating B cells but are relatively weak in inducing IFN-α and NK cell activation. Class B CpG oligonucleotides have been described, for example, in U.S. Pat. Nos. 6,194,388; 6,207,646; 6,214,806; 6,218,371; 6,239,116; and 6,339,068, the relevant disclosures therein are hereby incorporated by reference for the purpose or subject matter disclosed therein.

In some embodiments, a class B CpG oligonucleotide is represented by at least the formula:

5′X₁X₂CGX₃X₄3′,

in which X₁, X₂, X₃, and X₄ are nucleotides. In some embodiments, X₂ is adenine, guanine, or thymine. In some embodiments, X₃ is cytosine, adenine, or thymine.

In some embodiments, a class B CpG oligonucleotide is represented by at least the formula:

5′N₁X₁X₂CGX₃X₄N₂3′,

in which X₁, X₂, X₃, and X₄ are nucleotides and N is any nucleotide and N₁ and N₂ are nucleic acid sequences composed of from about 0-25 nucleotides each. In some embodiments, X₁X₂ is a dinucleotide selected from the group consisting of: GpT, GpG, GpA, ApA, ApT, ApG, CpT, CpA, CpG, TpA, TpT, and TpG; and X₃X₄ is a dinucleotide selected from the group consisting of: TpT, ApT, TpG, ApG, CpG, TpC, ApC, CpC, TpA, ApA, and CpA. In some embodiments, X₁X₂ is GpA or GpT and X₃X₄ is TpT. In some embodiments, X₁ or X₂ or both are purines and X₃ or X₄ or both are pyrimidines or X₁X₂ is GpA and X₃ or X₄ or both are pyrimidines. In some embodiments, X₁X₂ is a dinucleotide selected from the group consisting of: TpA, ApA, ApC, ApG, and GpG. In some embodiments, X₃X₄ is a dinucleotide selected from the group consisting of: TpT, TpA, TpG, ApA, ApG, GpA, and CpA. In some embodiments, X₁X₂ is a dinucleotide selected from the group consisting of: TpT, TpG, ApT, GpC, CpC, CpT, TpC, GpT and CpG; X₃ is a nucleotide selected from the group consisting of A and T and X₄ is a nucleotide, but wherein when X₁X₂ is TpC, GpT, or CpG, X₃X₄ is not TpC, ApT or ApC.

Class C CpG oligonucleotides, in some embodiments, contain at least two distinct motifs having unique and desirable stimulatory effects on cells of the immune system. In some embodiments, class C CpG oligonucleotides have both a traditional “stimulatory” CpG sequence and a “GC-rich” or “B-cell neutralizing” motif. Thus, in some embodiments, class C CpG oligonucleotides have immune stimulating effects that fall somewhere between those effects associated with class B CpG oligonucleotides, which are strong inducers of B cell activation and dendritic cell (DC) activation, and those effects associated class A CpG oligonucleotides which are strong inducers of IFN-α and natural killer (NK) cell activation but relatively poor inducers of B-cell and DC activation. Krieg AM et al. (1995) Nature 374:546-9; Ballas Z K et al. (1996) J Immunol 157:1840-5; Yamamoto S et al. (1992) J Immunol 148:4072-6. While typical class B CpG oligonucleotides often have phosphorothioate backbones and typical class A CpG oligonucleotides have mixed or chimeric backbones, typical C CpG oligonucleotides may have either stabilized, e.g., phosphorothioate, chimeric, or phosphodiester backbones, and in some embodiments, they have semi-soft backbones, e.g., a phosphodiester internucleotide linkage between the C and G nucleotides and other internucleotide linkages have a phosphorothioate linkage. Class C CpG oligonucleotides have been described, for example, in U.S. Pat. Nos. 7,566,703; 8,198,251; and 8,834,900, the relevant disclosures are hereby incorporated by reference.

In some embodiments, the stimulatory motif in a class C CpG oligonucleotide is defined by a formula: 5′ X₁DCGHX₂ 3′, wherein D is a nucleotide other than C, C is cytosine, G is guanine, H is a nucleotide other than G, and X₁ and X₂ are any nucleic acid sequence 0 to 10 nucleotides long. X₁ may include a CG, in which case there is preferably a T immediately preceding CG. In some embodiments DCG is TCG. X₁ is preferably from 0 to 6 nucleotides in length. In some embodiments, X₂ does not contain any poly G or poly A motifs. In some embodiments, class C CpG oligonucleotides have a poly-T sequence at the 5′ end or at the 3′ end. As used herein, “poly-A” or “poly-T” refers to a stretch of three or more consecutive A's or T's respectively, e.g., 5′ AAAA 3′ or 5′ TTTT 3′. As used herein, “poly-G” refers to a stretch of three or more consecutive G's, e.g., 5′ GGG 3′, occurring at the 5′ end or the 3′ end of a nucleic acid. In some embodiments, the B cell stimulatory domain of class C CpG oligonucleotides comprises TTTTTCG, TCG, TTCG, TTTCG, TTTTCG, TCGT, TTCGT, TTTCGT, or TCGTCGT.

In some embodiments, the “GC-rich” or “B-cell neutralizing” motif in a class C CpG oligonucleotide is referred to as either P or N and is positioned immediately 5′ to X₁ or immediately 3′ to X₂.

N is a B-cell neutralizing sequence that begins with a CGG trinucleotide and is at least 10 nucleotides long. A B-cell neutralizing motif includes at least one CpG sequence in which the CG is preceded by a C or followed by a G (Krieg AM et al. (1998) Proc Natl Acad Sci USA 95:12631-12636) or is a CG containing DNA sequence in which the C of the CG is methylated. Neutralizing motifs are motifs which has some degree of immunostimulatory capability when present in an otherwise non-stimulatory motif, but, which when present in the context of other immunostimulatory motifs serve to reduce the immunostimulatory potential of the other motifs.

P is a GC-rich palindrome containing sequence at least 10 nucleotides long. As used herein, “palindrome” and, equivalently, “palindromic sequence” refers to an inverted repeat, i.e., a sequence such as ABCDEE′D′C′B′A′ in which A and A′, B and B′, etc., are bases capable of forming the usual Watson-Crick base pairs. As used herein, “GC-rich palindrome” refers to a palindrome having a base composition of at least two-thirds G's and C's. In some embodiments the GC-rich domain is preferably 3′ to the “B cell stimulatory domain”. In the case of a 10-base long GC-rich palindrome, the palindrome thus contains at least 8 G's and C's. In the case of a 12-base long GC-rich palindrome, the palindrome also contains at least 8 G's and C's. In the case of a 14-mer GC-rich palindrome, at least ten bases of the palindrome are G's and C's. In some embodiments the GC-rich palindrome is made up exclusively of G's and C's.

In some embodiments the GC-rich palindrome has a base composition of at least 81% G's and C's. In the case of such a 10-base long GC-rich palindrome, the palindrome thus is made exclusively of G's and C's. In the case of such a 12-base long GC-rich palindrome, it is preferred that at least ten bases (83%) of the palindrome are G's and C's. In some preferred embodiments, a 12-base long GC-rich palindrome is made exclusively of G's and C's. In the case of a 14-mer GC-rich palindrome, at least twelve bases (86%) of the palindrome are G's and C's. In some preferred embodiments, a 14-base long GC-rich palindrome is made exclusively of G's and C's. The C's of a GC-rich palindrome can be unmethylated or they can be methylated.

In general this domain has at least 3 Cs and Gs, more preferably 4 of each, and most preferably 5 or more of each. The number of Cs and Gs in this domain need not be identical. It is preferred that the Cs and Gs are arranged so that they are able to form a self-complementary duplex, or palindrome, such as CCGCGCGG. This may be interrupted by As or Ts, but it is preferred that the self-complementarity is at least partially preserved as for example in the motifs CGACGTTCGTCG (SEQ ID NO: 2) or CGGCGCCGTGCCG (SEQ ID NO: 3). When complementarity is not preserved, it is preferred that the non-complementary base pairs be TG. In a preferred embodiment there are no more than 3 consecutive bases that are not part of the palindrome, preferably no more than 2, and most preferably only 1. In some embodiments the GC-rich palindrome includes at least one CGG trimer, at least one CCG trimer, or at least one CGCG tetramer.

In one example, the CpG oligonucleotide disclosed herein comprises the nucleotide sequence of SEQ ID NO: 1. Such a CpG oligonucleotide may contain one or more of chemical modifications known in the art or disclosed herein. In one example, the CpG oligonucleotide comprising the nucleotide sequence of SEQ ID NO:1 may contain phosphorothioate internucleotide linkages.

Any of the CpG oligonucleotides can be in dimeric form. In some instances, the dimeric form of CpG oligonucleotides can be a homodimer comprising two identical CpG oligonucleotide molecules. For example, the homodimer may comprise two CpG oligonucleotides, each of which comprises (e.g., consists of) SEQ ID NO:1. In other instances, the dimeric of CpG oligonucleotides may be a heterodimer comprising two different CpG oligonucleotide molecules. Such two CpG oligonucleotide molecules may differ in length, differ in nucleotide sequences, or both. Any of the dimeric CpG oligonucleotide complexes disclosed herein is also within the scope of the present disclosure.

The pharmaceutical composition disclosed herein may comprise any of the CpG oligonucleotide at a concentration of ≥400 μM, e.g., ≥500 μM, ≥600 μM, ≥700 μM, ≥800 M, ≥900 μM, ≥1,000 μM, ≥1,200 μM, ≥1,500 μM, ≥1,800 μM, ≥2,000 μM, ≥2,500 μM, ≥3,000 μM; ≥3,500 μM; ≥4,000 μM; ≥4,500 μM, or ≥5,000 μM. In some embodiments, the pharmaceutical composition disclosed herein may comprise any of the CpG oligonucleotide at a concentration of about 400 μM to about 5,000 μM, for example, about 500 μM to about 4,000 μM, about 500 μM to about 3,500 μM, about 500 μM to about 3,000 μM, about 500 μM to about 2,500 μM, about 500 μM to about 2,000 μM, about 500 μM to about 1,500 μM; about 500 μM to about 1,000 μM, or about 500 μM to about 800 μM.

(ii) Buffer agent

The CpG-containing pharmaceutical composition disclosed herein may further comprise a suitable buffer agent. A buffer agent is a weak acid or base used to maintain the pH of a solution near a chosen value after the addition of another acid or base. In some examples, the buffer agent disclosed herein can be a buffer agent capable of maintaining physiological pH despite changes in carbon dioxide concentration (produced by cellular respiration). Exemplary buffer agents include, but are not limited to a HEPES (4-(2-hydroxyethyl)-1-piperazineethanesulfonic acid) buffer, Dulbecco's phosphate-buffered saline (DPBS) buffer, or Phosphate-buffered Saline (PBS) buffer. Such buffers may comprise disodium hydrogen phosphate and sodium chloride, or potassium dihydrogen phosphate and potassium chloride.

The concentration of the buffer agent in the pharmaceutical composition described herein may range from about 20 mM to about 100 mM. For example, the concentration of the buffer agent may be about 20-30 mM, about 30-40 mM, about 30-50 mM, about 30-60 mM, about 30-70 mM, about 30-80 mM, about 30-90 mM, or about 30-100 mM. In some examples, the concentration of the buffer agent can be about 40 mM.

In general, the terms “about” and “approximately” mean within an acceptable error range for the particular value as determined by one of ordinary skill in the art. “About” can mean a range of less than ±30%, preferably less than ±20%, more preferably less than ±10%, more preferably less than ±5%, and more preferably still less than ±1% of a given value.

In some embodiments, the buffer agent in the pharmaceutical composition described herein may maintain a pH value of about 7-8. For example, the pH of the pharmaceutical composition can be about 7.0, 7.1, 7.2, 7.3, 7.4, 7.5, 7.6, 7.7, 7.8, 7.9, or 8.0. In one specific example, the pH of the pharmaceutical composition is about 7.5.

(iii) Salts

The pharmaceutical composition described herein comprises one or more suitable salts in a total concentration of about 80-130 mM. A salt is an ionic compound that can be formed by the neutralization reaction of an acid and a base. (Skoog, D. A; West, D. M.; Holler, J. F.; Crouch, S. R. (2004); Chapters 14-16. Fundamentals of Analytical Chemistry (8th ed.)). Salts are composed of related numbers of cations (positively charged ions) and anions (negative ions) so that the product is electrically neutral (without a net charge). An ion, as described herein, are atoms or molecules which have gained or lost one or more valence electrons giving the ion a net positive or negative charge. If the chemical species has more protons than electrons, it carries a net positive charge. If there are more electrons than protons, the species has a negative charge.

A cation (+), as described herein, is an ion with fewer electrons than protons, giving it a positive charge. (Douglas W. Haywick, (2007-2008). “Elemental Chemistry”). A cation with one positive charge can be called a monovalent cation; a cation with more than one positive charge can be called a polyvalent or multivalent cation. Non limiting examples of monovalent cations are hydrogen (H⁺), sodium (Na⁺), potassium (K⁺), ammonium (NH4⁺), Lithium (Li⁺), cuprous (Cu⁺), silver (Ag⁺), etc. Non limiting examples of multivalent cations are magnesium (Mg²⁺), calcium (Ca²⁺), barium (Ba²⁺), beryllium (Be²⁺), cupric (Cu²⁺), ferrous (Fe²⁺), ferric (Fe³⁺), lead(II) (Pb²⁺), lead(IV) (Pb⁴⁺), manganese(II) (Mn²⁺), strontium (Sr²⁺), tin(IV) (Sn⁴⁺), zinc (Zn²⁺), etc.

An anion, as described herein, is an ion with more electrons than protons, giving it a net negative charge. Non limiting examples of anions are azide (N₃⁻), bromide (Br⁻), chloride (Cl⁻), fluoride (F⁻), hydride (H⁻), iodide (I⁻), nitride (N⁻), Oxide (O²⁻), sulfide (S²⁻), carbonate (CO₃²⁻), hydrogen carbonate (HCO₃⁻), hydrogen sulfate (HSO⁴⁻), hydroxide (OH⁻), dihydrogen phosphage (H2PO₄⁻), sulfate (SO₄²⁻), sulfite (SO₃²⁻), silicate (SiO3²⁻), etc.

Suitable salts for use in the pharmaceutical compositions described herein may contain a monovalent cation and a monovalent or multi-valent anion. Alternatively, the salts for use in the pharmaceutical compositions described herein may contain a monovalent or multi-valent cation and a monovalent anion. Exemplary salts include, but are not limited to, potassium chloride (KCl), sodium chloride (NaCl), calcium chloride (CaCl₂)), Magnesium chloride (MgCl₂), Magnesium Sulfate(MgSO₄), Sodium Bicarbonate (NaHCO₃), Ammonium sulfate((NH₄)₂SO₄), calcium carbonate (Ca₂CO₃), or a combination thereof. In some embodiments, the pharmaceutical composition described herein comprises KCl, NaCl, CaCl₂), MgCl₂ or a combination thereof.

The total salt concentration in the pharmaceutical composition described herein may range from about 80 to about 130 mM, for example, about 80-120 mM, about 80-100 mM, about 80-90 mM, about 90-130 mM, about 100-130 mM, about 110-130 mM, or about 120-130 mM. In one specific example, the total salt concentration is about 120 mM.

The salts at the disclosed concentration range help maintain a majority of the CpG oligonucleotides contained therein in dimeric form. In some instances, more than 80% (e.g., 85%, 90%, 91%, 92%, 93%, 94%, 95%, 96%, 97%, 98%, 99% or above) of the CpG oligonucleotides in the composition are in dimeric form. Formation of dimeric CpG oligonucleotides can be measured by conventional methods or by the methods disclosed in the Examples below.

(iv) Other Components

The pharmaceutical composition described herein may further comprise a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some examples, the pharmaceutical composition described herein comprises liposomes containing any of the CpG oligonucleotides in dimeric form, which can be prepared by methods known in the art, such as described in Epstein, et al., Proc. Natl. Acad. Sci. USA 82:3688 (1985); Hwang, et al., Proc. Natl. Acad. Sci. USA77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The CpG oligonucleotides as described herein may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the CpG oligonucleotide, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. The CpG oligonucleotide-containing compositions may be placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets having a suitable size and can have a pH in the range of 5.5 to 8.0.

The emulsion compositions can be those prepared by mixing a CpG oligonucleotide with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### II. Therapeutic Applications

The pharmaceutical compositions disclosed herein, comprising CpG oligonucleotides in dimeric form, can be used to stimulate immune activity, for example, anti-tumor immune responses or anti-infectious immune responses.

To practice the method disclosed herein, an effective amount of any of the pharmaceutical compositions described herein can be administered to a subject (e.g., a human) in need of the treatment via a suitable route, such as intravenous administration, e.g., as a bolus or by continuous infusion over a period of time, by intratumoral, intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, a CpG oligonucleotide-containing pharmaceutical composition can be aerosolized using a fluorocarbon formulation and a metered dose inhaler, or inhaled as a lyophilized and milled powder. In some examples, the pharmaceutical composition described herein is formulated for intratumoral injection. In particular examples, the CpG oligonucleotide-containing pharmaceutical composition may be administered to a subject (e.g., a human patient) via a local route, for example, injected to a local site such as a tumor site or an infectious site.

As used herein, “an effective amount” refers to the amount of each active agent required to confer therapeutic effect on the subject, either alone or in combination with one or more other active agents. In some embodiments, the therapeutic effect is reduced tumor burden, reduction of cancer cells, or increased immune activity. Determination of whether an amount of CpG oligonucleotide achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of a CpG oligonucleotide in dimeric form may be appropriate. Various formulations and devices for achieving sustained release are known in the art.

In some example, dosages for a CpG oligonucleotide as described herein may be determined empirically in individuals who have been given one or more administration(s) of the CpG oligonucleotide. Individuals are given incremental dosages of the CpG oligonucleotide. To assess efficacy of the CpG oligonucleotide, an indicator of the disease/disorder can be followed.

Generally, for administration of any CpG oligonucleotide-containing pharmaceutical compositions described herein, an initial candidate dosage can be about 100 μg/kg of the CpG oligonucleotide. For the purpose of the present disclosure, a typical daily dosage might range from about any of 0.1 μg/kg to 1 μg/kg to 10 μg/kg to 100 μg/kg to 1 mg/kg to 2 mg/kg to 4 mg/kg to 40 mg/kg to 100 mg/kg or more, depending on the factors mentioned above. In some instances, the CpG oligonucleotide can be given to a subject (e.g., a human cancer patient) at a dosage of about 40 nmol to about 1,500 nmol, for example, about 50 nmol to about 1,000 nmol, about 50 nmol to about 800 nmol, about 50 nmol to about 500 nmol, about 50 nmol to about 300 nmol, about 50 nmol to about 200 nmol, or about 50 nmol to about 100 nmol.

For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg, or followed by a maintenance dose of about 1 mg/kg every other week.

However, other dosage regimens may be useful, depending on the pattern of pharmacokinetic decay that the practitioner wishes to achieve. For example, dosing from one-four times a week is contemplated. In some embodiments, dosing ranging from about 100 μg/mg to about 4 mg/kg of a CpG oligonucleotide-containing pharmaceutical composition as described herein (such as about 1 μg/kg, about 10 μg/kg, about 30 μg/kg, about 100 μg/kg, about 300 μg/kg, about 1 mg/kg, about 2 mg/kg and about 4 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen of the CpG oligonucleotide used) can vary over time.

In some embodiments, for an adult patient of normal weight, doses ranging from about 0.3 to 5.0 mg/kg may be administered. The particular dosage regimen, i.e., dose, timing and repetition, will depend on the particular individual and that individual's medical history, as well as the properties of the individual agents (such as the half-life of the agent, and other considerations well known in the art).

In some embodiments, the method described herein comprises administering to a subject in need of the treatment (e.g., a human patient) one or multiple doses of the CpG oligonucleotide-containing pharmaceutical composition.

For the purpose of the present disclosure, the appropriate dosage CpG oligonucleotide as described herein will depend on the specific CpG oligonucleotide, the type and severity of the disease/disorder, the CpG oligonucleotide is administered for preventive or therapeutic purposes, previous therapy, the patient's clinical history and response to the CpG oligonucleotide, and the discretion of the attending physician. A clinician may administer a CpG oligonucleotide, until a dosage is reached that achieves the desired result. In some embodiments, the desired result is a decrease in tumor burden, a decrease in cancer cells, or increased immune activity. Methods of determining whether a dosage resulted in the desired result would be evident to one of skill in the art. Administration of one or more CpG oligonucleotides can be continuous or intermittent, depending, for example, upon the recipient's physiological condition, whether the purpose of the administration is therapeutic or prophylactic, and other factors known to skilled practitioners. The administration a CpG oligonucleotide may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In some embodiments, a CpG oligonucleotide-containing pharmaceutical composition as described herein are administered to a subject in need of the treatment at an amount sufficient to reduce tumor burden or cancer cell growth, by at least 5% (e.g., 10%, 20%, 30%, 40%, 50%, 60%, 70%, 80%, 90% or greater) in vivo. In other embodiments, the CpG oligonucleotide-containing pharmaceutical compositions as described herein can be administered in an amount effective in increasing immune activity by at least 5% (e.g., 10%, 20%, 30%, 40%, 50%, 60%, 70%, 80%, 90% or greater).

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularlly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble CpG oligonucleotide-containing compositions as described herein can be administered by the drip method, whereby a pharmaceutical formulation containing the CpG oligonucleotide in dimeric form and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the CpG oligonucleotide, can be dissolved and administered in a pharmaceutical excipient such as Water-for-Injection, 0.9% saline, or 5% glucose solution.

In some embodiments, a CpG oligonucleotide-containing pharmaceutical composition as described herein can be administered via site-specific or targeted local delivery techniques. Examples of site-specific or targeted local delivery techniques include various implantable depot sources of the CpG oligonucleotide-containing composition or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an oligonucleotide can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

The subject to be treated by the methods described herein can be a mammal, such as a farm animals, sport animals, pets, primates, horses, dogs, cats, mice and rats. In one example, the subject is a human. The CpG oligonucleotide-containing composition as described herein may be used for enhancing immune activity, for example, T cell activity, in a subject in need of the treatment.

In some examples, the subject may be a human patient having, suspected of having, or at risk for a cancer. Non limiting examples of cancers can be squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulvar cancer, thyroid cancer, hepatic carcinoma, gastric cancer, melanoma, and various types of head and neck cancer, including squamous cell head and neck cancer. In some embodiments, the cancer can be lung cancer, melanoma, colorectal cancer, renal-cell cancer, urothelial carcinoma, or Hodgkin's lymphoma.

In other examples, the subject may be a human patient having, or suspected of having or at risk for an infectious disease, which are associated with various pathogenic microorganisms.

The pathogenic microorganisms can be bacteria, fungi, parasites or viruses. Non-limiting examples of pathogenic microorganisms to cause infectious diseases can be Bordetella pertussis, Candida albicans, Chlamydia trachomatis, Escherichia coli, Neisseria gonorrhoeae, Neisseria meningitides, Gardnerella vaginalis, Haemophilus ducreyi, Lactobacillus crispatus, Lactobacillus gasseri, Mycobacterium bovis, Mycoplasma hominis, Mycoplasma genitalium, Treponema pallidum, Ureaplasma urealyticum, Yersinia pestis, Human papilloma virus (HPV), Hepatitis B virus (HBV), Epstein-Barr virus (EBV), Polyomavirus, Pseudomonas aeruginosa, Leishmania, and Toxoplasma gondii.

A subject having a target disease or disorder (e.g., cancer or an infectious disease) can be identified by routine medical examination, e.g., laboratory tests, organ functional tests, CT scans, or ultrasounds. A subject suspected of having any of such target disease/disorder might show one or more symptoms of the disease/disorder. A subject at risk for the disease/disorder can be a subject having one or more of the risk factors associated with that disease/disorder. Such a subject can also be identified by routine medical practices.

The particular dosage regimen, i.e., dose, timing and repetition, used in the method described herein will depend on the particular subject (e.g., a human patient) and that subject's medical history.

Treatment efficacy for a target disease/disorder can be assessed by, e.g., a method described in the Examples below.

In some embodiments, a CpG oligonucleotide-containing pharmaceutical composition may be co-used with another suitable therapeutic agent (e.g., an anti-cancer agent an anti-viral agent, or an anti-bacterial agent) and/or other agents that serve to enhance and/or complement the immunostimulatory effect of CpG oligonucleotide. In such combined therapy, the CpG oligonucleotide-containing composition and the additional therapeutic agent (e.g., an anti-cancer therapeutic agent or others described herein) may be administered to a subject in need of the treatment in a sequential manner, i.e., each therapeutic agent is administered at a different time. Alternatively, these therapeutic agents, or at least two of the agents, are administered to the subject in a substantially simultaneous manner.

Sequential or substantially simultaneous administration of each agent can be affected by any appropriate route including, but not limited to, oral routes, intravenous routes, intramuscular, subcutaneous routes, and direct absorption through mucous membrane tissues. The agents can be administered by the same route or by different routes. For example, a first agent (e.g., a CpG oligonucleotide-containing pharmaceutically composition as described herein)) can be administered intratumorally, and a second agent (e.g., an anti-cancer agent) can be administered intravenously or orally.

As used herein, the term “sequential” means, unless otherwise specified, characterized by a regular sequence or order, e.g., if a dosage regimen includes the administration of a CpG oligonucleotide-containing composition and an anti-cancer agent, a sequential dosage regimen could include administration of the CpG oligonucleotide before, simultaneously, substantially simultaneously, or after administration of the anti-cancer agent, but each agent will be administered in a regular sequence or order.

The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents described herein.

Combination therapy can also embrace the administration of the agents described herein (e.g., a CpG oligonucleotide-containing pharmaceutical composition and an anti-cancer agent) in further combination with other biologically active ingredients (e.g., a different anti-cancer agent) and non-drug therapies (e.g., surgery).

It should be appreciated that any combination of a CpG oligonucleotide-containing composition and another anti-cancer agent (e.g., a chemotherapeutic agent) may be used in any sequence for treating a cancer. The combinations described herein may be selected on the basis of a number of factors, which include but are not limited to the effectiveness of reducing tumor formation or tumor growth, reducing cancer cells, increasing immune activity, and/or alleviating at least one symptom associated with the cancer, or the effectiveness for mitigating the side effects of another agent of the combination. For example, a combined therapy described herein may reduce any of the side effects associated with each individual members of the combination, for example, a side effect associated with the anti-cancer agent.

In some embodiments, another anti-cancer therapy is a chemotherapy, a radiation therapy, a surgical therapy and/or an immunotherapy. Examples of the chemotherapeutic agents include, but are not limited to, Carboplatin or Cisplatin, Docetaxel, Gemcitabine, Nab-Paclitaxel, Paclitaxel, Pemetrexed, and Vinorelbine. Examples of radiation therapy include, but are not limited to, ionizing radiation, gamma-radiation, neutron beam radiotherapy, electron beam radiotherapy, proton therapy, brachytherapy, systemic radioactive isotopes and radiosensitizers. Examples of a surgical therapy include, but are not limited to, a curative surgery (e.g., tumor removal surgery), a preventive surgery, a laparoscopic surgery, and a laser surgery. Examples of an immunotherapy include, but are not limited to, adoptive cell transfer and therapeutic cancer vaccines.

Additional examples of chemotherapy include, but are not limited to, platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin HCl, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives); Purine antagonists (Thioguanine, Fludarabine, Cladribine, 6-Mercaptopurine, Pentostatin, clofarabine and relatives) and Pyrimidine antagonists (Cytarabine, Floxuridine, Azacitidine, Tegafur, Carmofur, Capacitabine, Gemcitabine, hydroxyurea, 5-Fluorouracil (5FU), and relatives); Alkylating agents, such as Nitrogen mustards (e.g., Cyclophosphamide, Melphalan, Chlorambucil, mechlorethamine, Ifosfamide, Trofosfamide, Prednimustine, Bendamustine, Uramustine, Estramustine, and relatives); nitrosoureas (e.g., Carmustine, Lomustine, Semustine, Fotemustine, Nimustine, Ranimustine, Streptozocin, and relatives); Triazenes (e.g., Dacarbazine, Altretamine, Temozolomide, and relatives); Alkyl sulphonates (e.g., Busulfan, Mannosulfan, Treosulfan, and relatives); Procarbazine; Mitobronitol, and Aziridines (e.g., Carboquone, Triaziquone, ThioTEPA, triethylenemalamine, and relatives); Antibiotics, such as Hydroxyurea, Anthracyclines (e.g., doxorubicin agent, daunorubicin, epirubicin and other derivatives); Anthracenediones (e.g., Mitoxantrone and relatives); Streptomyces family (e.g., Bleomycin, Mitomycin C, Actinomycin, Plicamycin); and Ultraviolet light.

### III. Kits for Use in Modulating Immune Responses

The present disclosure also provides kits for use in modulating (e.g., enhancing) immune activity (e.g., T cell activity), alleviating cancer (e.g., lung cancer, melanoma, colorectal cancer, or renal-cell cancer), and/or treating or reducing the risk for cancer. Such kits can include one or more containers comprising a CpG oligonucleotide-containing pharmaceutical composition, e.g., any of those described herein.

In some embodiments, the kit can comprise instructions for use in accordance with any of the methods described herein. For example, the included instructions can comprise a description of administration of the CpG oligonucleotide-containing composition to treat, delay the onset, or alleviate a target disease as those described herein. The kit may further comprise a description of selecting an individual suitable for treatment based on identifying whether that individual has the target disease. In still other embodiments, the instructions comprise a description of administering the CpG oligonucleotide-containing composition to an individual at risk of the target disease.

The instructions relating to the use of a CpG oligonucleotide-containing composition generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder associated with cancer, such as those described herein. Instructions may be provided for practicing any of the methods described herein.

The kits as described herein are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a CpG oligonucleotide-containing composition such as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### IV. General Techniques

The practice of the present invention will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Preparation and Evaluation of CpG Oligonucleotide MBS513

MBS513 is a single strand 25 bp CpG oligodeoxynucleotide (CpG DNA) having a nucleotide sequence of SEQ ID NO: 1 and phosphorothioate internucleotide linkages. MBS513 stock solution was prepared by dissolving the CpG DNA powder in filtered distilled deionized water (ddH₂O) at 15 mg/ml (1.87 mM). MBS513 stock solution was stored at −20° C. until future use. To obtain MBS513 working solution, the stock solution was further diluted in SELEX buffer (40 mM HEPES, 5 mM KCl, 111 mM NaCl, 1 mM CaCl₂), 1 mM MgCl₂, pH 7.5) to the desired concentrations for subsequent experiments.

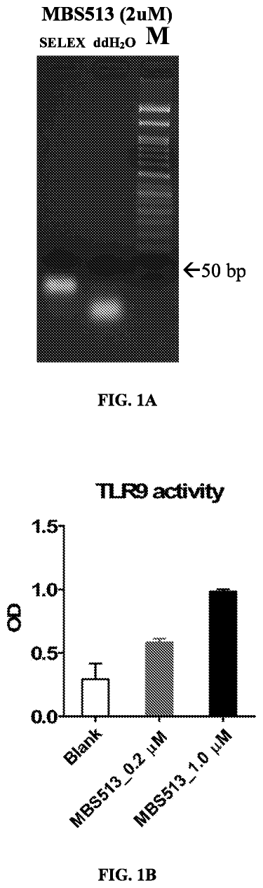

To compare the state of MBS513 in solutions, MBS513 was dissolved in either ddH2O or SELEX buffer at 2 μM. The DNAs were visualized on an agarose gel to determine the amount of MBS513 in monomeric or dimeric form in either solution. As shown in FIG. 1A, MBS513 dissolved in SELEX buffer ran on the gel at the same level of the 50 bp marker, indicating MBS513 in SELEX buffer were predominantly in dimeric form. By contrast, MBS513 dissolved in ddH2O were mostly in monomeric form, which ran faster on the agarose gel than the MBS513 dissolved in SELEX buffer. HPLC analysis of MBS513 in either SELEX buffer or ddH₂O obtained similar results; only 10% of MBS513 in SELEX buffer were monomers, whereas 100% of MBS513 in ddH₂O were monomers.

Further, the function of MBS513 was tested in a TLR9 activation assay using HEK-Blue™ hTLR9 cells (InvivoGen). HEK-Blue™-hTLR9 cells express human TLR9 and an inducible SEAP (secreted embryonic alkaline phosphatase) reporter. The SEAP gene is placed under the control of the IFN-β minimal promoter fused to five NF-κB and AP-1-binding sites. Stimulation with a TLR9 ligand activates NF-κB and AP-1 which induce the production of SEAP. Levels of SEAP can be easily observed by naked eye and can be quantified by reading the Optical Density (OD) using a spectrophotometer at 620-655 nm. MBS513 was added to a flat-bottom 96-well plate at 20 μl per well. The wells that were plated with 20 μl per well of sterile, endotoxin-free ddH₂O were included as negative control. HEK-Blue™ hTLR9 cells were removed from the culture, rinsed and dissociated to obtain single cell suspension at 450,000 cells per ml in pre-warmed PBS. 180 μl of the cell suspension (about 80,000 cells) were added into each well of the 96-well plate that contained MBS513 or ddH₂O. The final volume of the reaction is 200 μl and the final concentrations of MBS513 tested in the reaction are 0.2 μM and 1 μM. The cells were then incubated at 37° C. in 5% CO2 for 6-16 hours. The amount of SEAP produced represented the extent of TLR9 activation. FIG. 1B shows that MBS513 activates TLR9 signaling in a dose-dependent manner.

Results obtained from this Example indicate that salts, optionally other components in a suitable solution, can maintain a majority of CpG oligonucleotides in dimeric form.

### Example 2: Effect of MBS513 in Inhibiting Tumor Growth In Vivo

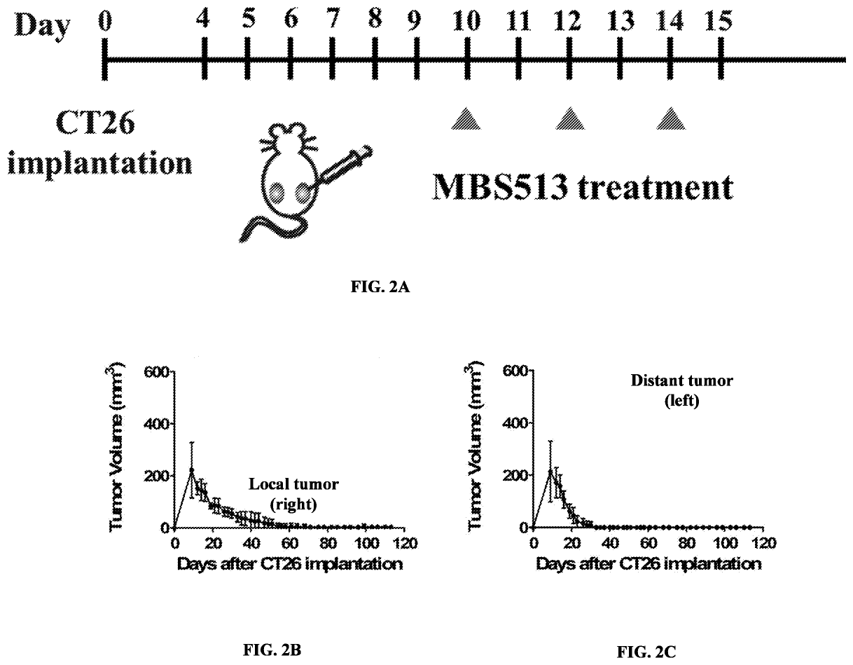

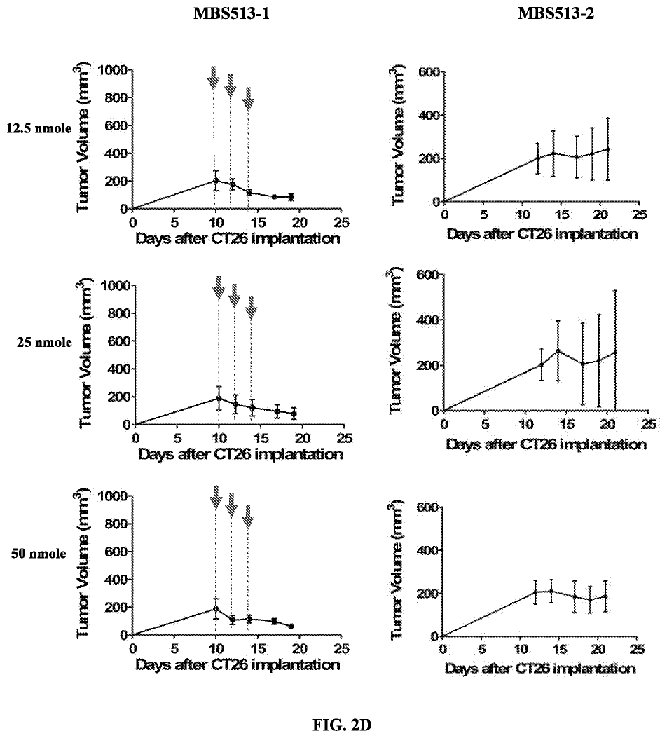

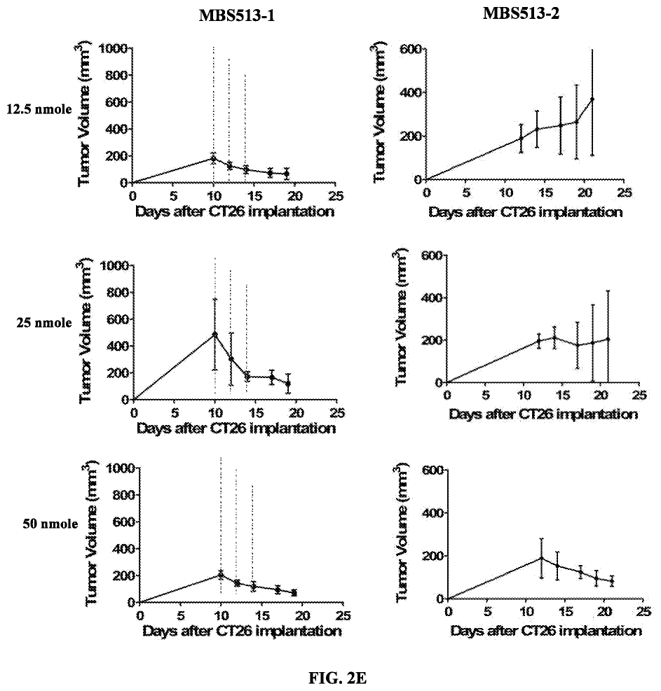

To evaluate the effect of MBS513 in tumor growth in vivo, the following experiments, as illustrated in FIG. 2A, were designed. 5×10⁵ CT26 tumor cells were injected subcutaneously on both the right and left side of the abdomen of the BALB/c mice. When tumor size reached 200 cm³, 12.5 nmol, 25 nmole, or 50 nmol of MBS513 were injected into the tumor only on the right side of the animals in 50 μl volume 10, 12, and 14 days after tumor implantation. Tumor volume on both sides of the abdomen was measured every 2 to 3 days. Mice were sacrificed when tumor volume reached 25,000 cm³ in accordance with the guidelines. As shown in FIG. 2B and FIG. 2C, MBS513 inhibited the growth not only of the local tumors (right side), but also of the distant tumors on the other side of the abdomen (left side). High doses of MBS513, e.g., 50 nmol, showed better effectiveness of inducing systemic immune responses leading to tumor suppression. FIG. 2D.

To further validate the distant tumor inhibition effect of MBS513, two different batches of MBS513 were tested (MBS513-1 and MBS513-2). A similar experiment was carried out as described above, and various doses of MBS513 were used to treat the mice carrying the tumors (12.5 nmol, 25 nmol, and 50 nmol). Interestingly, MBS513-1 showed better tumor inhibition effect to both the local tumor and the distant tumor compared to MBS513-2. FIG. 2D and FIG. 2E.

To ascertain the contributing factor of the difference in tumor inhibition between the two batches of MBS513, MBS513-1 and MBS513-2 were compared by HPLC analysis. 200 μg of MBS513-1 and MBS513-2 were dissolved in 50 μl of SELEX buffer and subjected to HPLC profiling, respectively. Table 1 shows that MBS513-1 contains 85.7% of dimers and 14.2% of monomers, whereas MBS513-2 contains 73.3% of dimers and 11.1% (AU*min/AU*min) of monomers, These results indicate that the higher dimer content in MBS513-1 contributes to better efficacy in inhibiting both the local tumor and the distant tumor.

TABLE 1
Contents of Dimer and Monomer in Two Batches of MBS513

| | MBS513-1 | MBS513-2 |
|---|---|---|
| Dimer | 85.7% | 73.3% |
| Monomer | 14.2% | 11.1% |

Similarly, the tumor inhibition effect of MBS513-1 on local and distant tumors was compared to SD-101, which is a CpG DNA currently being evaluated in clinical trials. The effect of SD-101 was evaluated in Sagiv-Barfi et al., Science Translational Medicine, 31 Jan. 2018: Vol. 10, Issue 426, eaan4488. The results indicate that SD-101 can reduce the size of local tumor, but failed to elicit tumor inhibition effect on the distant tumors. By contrast, MBS513-1, was able to reduce tumor volume of both the local and distant tumors.

In sum, the results obtained from this Examiner indicate that the CpG oligonucleotides in dimeric form showed better anti-tumor effects relative to the monomeric counterpart. More surprisingly, local injection of the dimeric CpG oligonucleotides induced systemic anti-tumor immune responses, leading to reduced volume of distant tumor not subject to injection of the CpG oligonucleotide.

### Example 3: Formation of Dimeric CpG Oligonucleotides is Concentration-Dependent

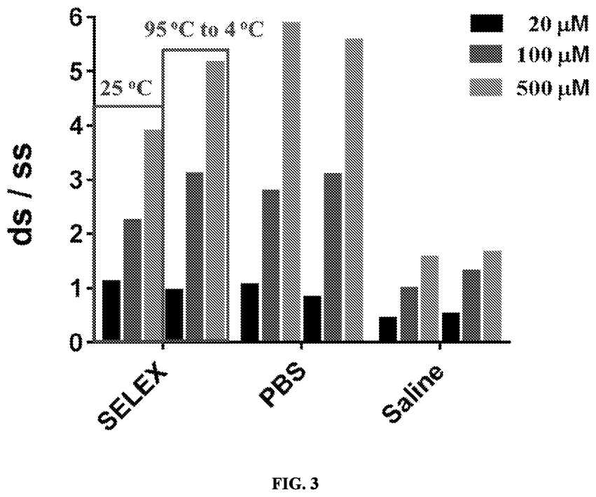

MBS513 was dissolved in SELEX buffer, PBS buffer, or saline at different concentrations, including 20 μM, 100 μM, and 500 μM. The solutions thus formed were incubated at various temperatures as indicated (FIG. 3) and the contents of dimeric MBS513 and monomeric MBS513 in the solutions were analyzed by HPLC. The ratio between dimeric MBS513 and monomeric MBS513 (ds/ss) was calculated by the dimer area (AU*min)/the monomer area (AU*min) as determined by HPLC.

As shown in FIG. 3, the formation of dimeric CpG comlex is concentration-dependent. The CpG oligonucleotides in SELEX buffer and PBS buffer showed similar dd/ss ratios.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A pharmaceutical composition, comprising:
(i) a CpG oligonucleotide;
(ii) a buffering agent; and
(iii) one or more salts at a total concentration of about 80-130 mM;
wherein at least 80% of the CpG oligonucleotide in the composition is in dimeric form.

2. The pharmaceutical composition of claim 1, wherein the CpG oligonucleotide comprises the nucleotide sequence of 5′-TCGAACGTTCGAACGTTCGAACGTT-3′ (SEQ ID NO: 1).

3. The pharmaceutical composition of claim 1, wherein the CpG oligonucleotide is a modified oligonucleotide.

4. The pharmaceutical composition of claim 3, wherein the modified oligonucleotide comprises one or more phosphorothioate internucleotide linkage, a methylphosphonate linkage, or a boranophosphate linkage.

5. The pharmaceutical composition of claim 1, wherein the buffering agent is HEPES, DPBS, or PBS buffer.

6. The pharmaceutical composition of claim 1, wherein the concentration of the buffering agent in the composition is 30-60 mM.

7. The pharmaceutical composition of claim 1, wherein the pharmaceutical composition has a pH of 7-8.

8. The pharmaceutical composition of claim 1, wherein the total concentration of salt is about 120 mM.

9. The pharmaceutical composition of claim 1, wherein the one or more salts comprise KCl, NaCl, CaCl₂, MgCl₂, or a combination thereof.

10. The pharmaceutical composition of claim 1, wherein the pharmaceutical composition comprises the CpG oligodeoxynucleotide at a concentration of at least about 500 μM.

11. The pharmaceutical composition of claim 10, wherein the CpG oligodeoxynucleotide is at a concentration of about 500 μM to 5,000 μM.

12. A dimeric oligonucleotide complex, which comprises two CpG oligonucleotide molecules, at least one of which comprises the nucleotide sequence of SEQ ID NO:1.

13. The dimeric oligonucleotide complex of claim 12, wherein both of the two CpG oligonucleotide molecules comprise the nucleotide sequence of SEQ ID NO:1.

14. A method for stimulating an immune response in a subject, comprising:
administering to a subject in need thereof an effective amount of the pharmaceutical composition of claim 1 or a dimeric oligonucleotide complex comprising two CpG oligonucleotide molecules, at least one of which comprises the nucleotide sequence of SEQ ID NO:1.

15. The method of claim 14, wherein the subject is a human patient having or suspected of having a cancer or an infectious disease.

16. The method of claim 15, wherein the human patient has cancer, which is selected from the group consisting of melanoma, colon cancer, lung cancer, breast cancer, liver cancer, and lymphoma.

17. The method of claim 14, wherein the pharmaceutical composition or the dimeric oligonucleotide complex is administered to the subject at a dosage of 100 μg/kg to 4,000 μg/kg of the CpG oligonucleotide.

18. The method of claim 14, wherein the pharmaceutical composition or the dimeric oligonucleotide complex is administered to the subject at a dosage of about 40 nmol to about 150 nmol.

19. The method of claim 18, wherein the pharmaceutical composition or the dimeric oligonucleotide complex is administered to the subject at a dosage of about 50 nmol.

20. The method of claim 14, wherein the pharmaceutical composition is administered to the subject by intratumoral injection.
